(19) United States
(12) Patent Application Publication
(10) Pub. No.: US 2026/0030936 A1
(43) Pub. Date: Jan. 29, 2026

(54) DRIVER MONITORING DEVICE, DRIVER MONITORING METHOD, AND STORAGE MEDIUM

(71) Applicant: HONDA MOTOR CO., LTD., Tokyo (JP)

(72) Inventors: Satoru Shinkawa, Wako-shi (JP); Shigenobu Mitsuzawa, Wako-shi (JP); Hiroshi Ono, Wako-shi (JP); Hiroki Taniguchi, Wako-shi (JP); Nozomi Nagatani, Wako-shi (JP)

(21) Appl. No.: 19/211,666

(22) Filed: May 19, 2025

(30) Foreign Application Priority Data
Jul. 26, 2024 (JP) 2024-121018

(51) Int. Cl. G07C 5/04; A61B 5/024; G01C 21/36

(52) U.S. Cl. CPC G07C 5/04; A61B 5/024; G01C 21/3691; A61B 2503/22

(57) ABSTRACT

A driver monitoring device includes: a biometric information recognition portion that recognizes biometric information of a driver of a moving body; a driving behavior recognition portion that recognizes driving behavior of the driver, who is driving the moving body; a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.

### INCORPORATION BY REFERENCE

The present application claims priority under 35 U.S.C. § 119 to Japanese Patent Application No. 2024-121018 filed on Jul. 26, 2024. The content of the application is incorporated herein by reference in its entirety.

### BACKGROUND OF THE INVENTION

### Field of the Invention

The present invention relates to a driver monitoring device, a driver monitoring method, and a storage medium.

### Description of the Related Art

In recent years, approaches have been actively made to provide access to sustainable transportation systems that take into consideration even vulnerable traffic participants. To achieve this, efforts have been focused on research and development into preventive safety technology to further improve traffic safety and convenience.

For example, a technique is disclosed in Japanese Patent Laid-Open No. 2019-61480 that calculates a drowsiness risk of a driver based on information obtained from the driver of a vehicle, calculates a monotonous driving risk based on information regarding a travel route of the vehicle, and estimates a time at which a future drowsiness level of the driver exceeds a predetermined threshold, based on the drowsiness risk and the monotonous driving risk.

In the preventive safety technology, it is effective for the driver, who is driving a moving body, to predict changes in the degree of risk of a driving situation with which the driver is confronted and to prevent occurrence of accidents of the moving body beforehand. Therefore, an object of the present application is to calculate an index that predicts future changes in the risk of the driving situation with which the driver is confronted.

In order to solve the above problems, the present application aims to calculate an index that predicts future changes in risk of a driving situation with which the driver is confronted. Thus, this further improves traffic safety and contributes to the development of a sustainable transportation system.

### SUMMARY OF THE INVENTION

A first aspect for achieving the above object provides a driver monitoring device including: a biometric information recognition portion that recognizes biometric information of a driver of a moving body; a driving behavior recognition portion that recognizes driving behavior of the driver, who is driving the moving body; and a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.

In the driver monitoring device, the driver monitoring device may further include a health level recognition portion that recognizes a health level of the driver, based on pre-driving biometric information, which is the biometric information of the driver, recognized by the biometric information recognition portion before the driver starts driving the moving body, and the predicted risk score calculation portion may calculate the predicted risk score, based on the driving behavior of the driver recognized by the driving behavior recognition portion at the predetermined time point, the on-driving biometric information recognized by the biometric information recognition portion at the predetermined time point and the health level of the driver recognized by the health level recognition portion.

In the driver monitoring device, the biometric information recognition portion may recognize, as the biometric information of the driver, a heart rate of the driver.

In the driver monitoring device, the driver monitoring device may further include: a moving body position recognition portion that recognizes a position of the moving body; and a moving environment recognition portion that recognizes a moving environment of the moving body after the predetermined time point, based on the position of the moving body at the predetermined time point recognized by the moving body position recognition portion, and the predicted risk score calculation portion may compensate the predicted risk score based on the moving environment estimated by a moving environment estimation portion.

In the driver monitoring device, the driver monitoring device may further include a moving route guidance portion that guides a moving route that is a moving environment recognized by the moving environment recognition portion to reduce a driving load of the driver when the predicted risk score calculation portion calculates the predicted risk score that is equal to or greater than a predetermined value.

A second aspect for achieving the above object provides, in the driver monitoring device, a driver monitoring method to be executed by a computer, the driver monitoring device including: a biometric information recognition step of recognizing biometric information of a driver of a moving body; a driving behavior recognition step of recognizing driving behavior of the driver, who is driving the moving body; and a predicted risk score calculation step of calculating, based on driving behavior of the driver recognized in the driving behavior recognition step at a predetermined time point while the driver is driving the moving body and on-driving biometric information, which is biometric information of the driver, recognized in the biometric information recognition step at the predetermined time point, a predicted risk score indicating a risk level regarding movement of the moving body due to driving of the driver after the predetermined time point.

A third aspect of for achieving the above object provides a non-transitory computer-readable storage medium storing a program causing a computer to function as: a biometric information recognition portion that recognizes biometric information of a driver of a moving body; a driving behavior recognition portion that recognizes driving behavior of the driver, who is driving the moving body; and a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.

According to the driver monitoring device, the driver monitoring method, and the storage medium, it is possible to calculate an index that predicts future changes in risk of a driving situation with which the driver is confronted.

### BRIEF DESCRIPTION OF THE DRAWINGS

FIG. 1 is an explanatory diagram of a usage mode of a driver monitoring device;

FIG. 2 is a configuration diagram of the driver monitoring device;

FIG. 3 is an explanatory diagram illustrating a corresponding relationship between life habit data, driving-related abilities, and driving diagnosis indices for coaching;

FIG. 4 is a flowchart of a process calculating a predicted risk score; and

FIG. 5 is an explanatory diagram of a concept of a predicted risk score.

### DETAILED DESCRIPTION OF THE INVENTION

### 1. Usage Mode of Driver Monitoring Device

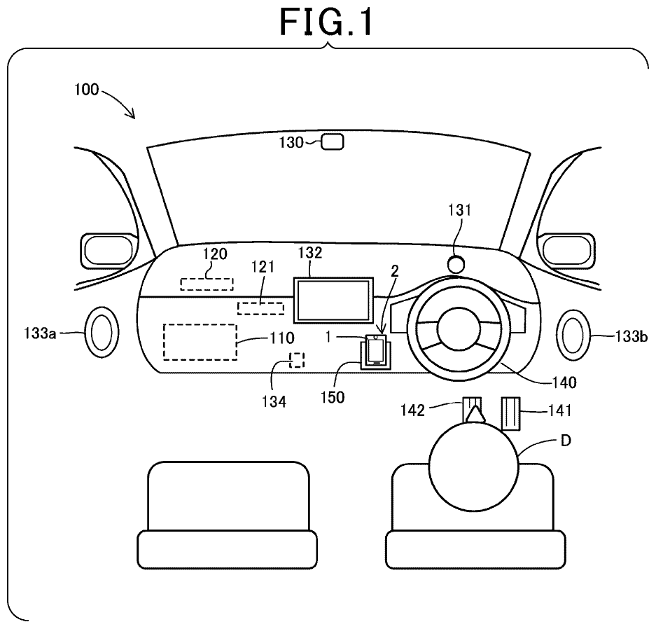

A usage mode of a driver monitoring device 2 according to the present embodiment will be described with reference to FIG. 1. The driver monitoring device 2 is configured as a function of a communication terminal 1 used by a driver D of a vehicle 100. The communication terminal 1 is a smartphone, a mobile phone, a tablet terminal, or the like. The communication terminal 1 is attached to a terminal holder 150 provided in the vehicle 100. The vehicle 100 corresponds to a moving body of the present disclosure. The moving body of the present disclosure may be an aircraft, a ship, or the like other than the vehicle.

The vehicle 100 includes, for example, an electronic control unit (ECU) 110 that controls an operation of the vehicle 100, a communication unit 120, a navigation device 121, a front camera 130 that captures an image of a front side of the vehicle 100, a driver monitor camera 131 that captures an image of the driver D, a display 132, speakers 133a and 133b, and an acceleration sensor 134. The ECU 110 communicates with the communication terminal 1 via the communication unit 120. The vehicle 100 includes, as driving operation portions, a steering wheel 140, an accelerator pedal 141, and a brake pedal 142.

The driver monitoring device 2 recognizes the operation (driving behavior) of the steering wheel 140, the accelerator pedal 141, and the brake pedal 142 by the driver D by receiving operation information through communication with the ECU 110, or by detection information from the acceleration sensor built into the communication terminal 1. The driver monitoring device 2 acquires biometric information (for example, heart rate) of the driver D measured by a wearable device worn by the driver D, through communication with the wearable device.

Then, the driver monitoring device 2 calculates, based on the driving behavior and the biometric information of the driver D, a predicted risk score indicating a risk level for the movement of the vehicle 100 by the driving of the driver D after a predetermined point of time when the driving behavior and the biometric information are recognized. The higher the risk level, the larger the value of the predicted risk score. Furthermore, the driver monitoring device 2 gives a notification to the driver D according to the level of the predicted risk score and supports the movement of the vehicle 100 by the driving of the driver D.

### 2. Configuration of Driver Monitoring Device

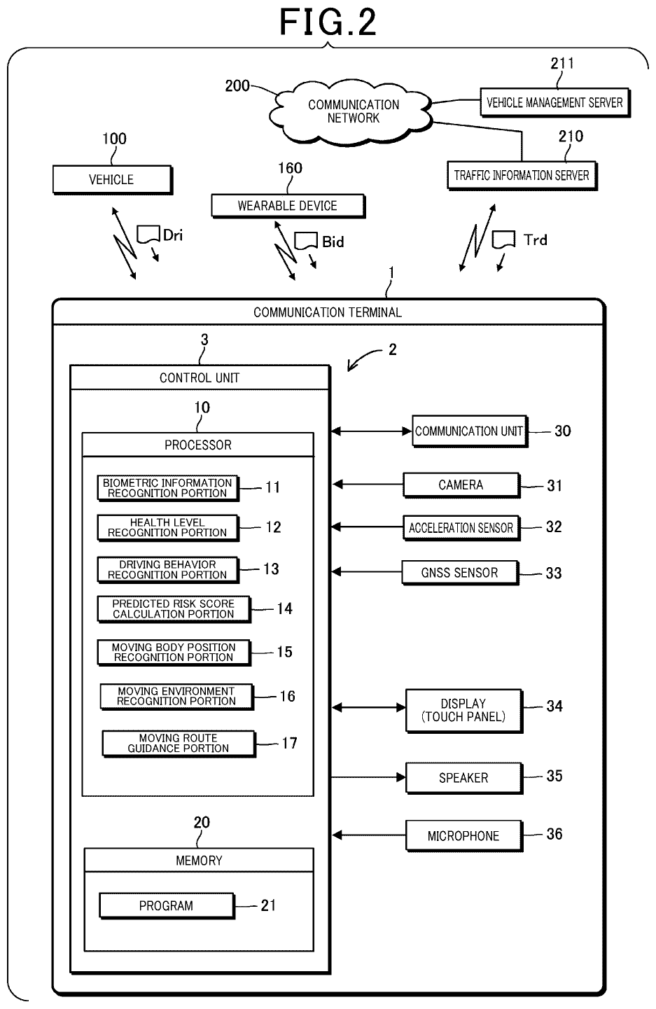

A configuration of the driver monitoring device 2 will be described with reference to FIG. 2. The communication terminal 1 having functions of the driver monitoring device 2 includes a control unit 3 including a processor 10 and a memory 20, a communication unit 30, a camera 31, an acceleration sensor 32, a global navigation satellite system (GNSS) sensor 33, a display 34, a speaker 35, and a microphone 36.

The communication unit 30 includes a transmitter and a receiver and performs near-field wireless communication between the vehicle 100 (specifically, the ECU 110 mounted on the vehicle 100) and a wearable device 160 (for example, a smart watch, wearable smart underwear, or a sensing device attached to the body or skin) worn by the driver D, using specifications of Bluetooth (registered trademark) and UWB (Ultra-Wide Band). The communication unit 30 communicates with a traffic information server 210, a vehicle management server 211, and the like via a communication network 200.

The camera 31 captures an image of the driver D in a state where the communication terminal 1 is attached to the terminal holder 150 (see FIG. 1). The acceleration sensor 32 detects, for example, six axes (acceleration in each of front-rear, left-right, and up-down axial directions, and an angular velocity of each axis). The GNSS sensor 33 detects a position of the communication terminal 1. The position detected by the GNSS sensor 33 in a state where the communication terminal 1 is attached to the terminal holder 150 becomes a current position of the vehicle 100. The display 34 is a touch panel, and the driver D touches the display 34 to instruct the driver monitoring device 2 to use or stop functions thereof.

The memory 20 stores an application program 21 of the driver monitoring device 2. The Processor 10 reads and executes the program 21, and thus functions as a biometric information recognition portion 11, a health level recognition portion 12, a driving behavior recognition portion 13, a predicted risk score calculation portion 14, a moving body position recognition portion 15, a moving environment recognition portion 16, and a moving route guidance portion 17.

A process to be executed by the biometric information recognition portion 11 corresponds to a biometric information recognition step in a driver monitoring method of the present disclosure, and a process to be executed by the driving behavior recognition portion 13 corresponds to a driving behavior recognition step in the driver monitoring method of the present disclosure. A process to be executed by the predicted risk score calculation portion 14 corresponds to a predicted risk score calculation step of the present disclosure.

The biometric information recognition portion 11 acquires, through communication with the wearable device 160, measurement information Bid related to a physical activity of the driver D measured by the wearable device 160, and thus recognizes biometric information of the driver D. The biometric information involves a heart rate, a sleep time, a quality of sleep (REM sleep, non-REM sleep, or a depth of sleep), an exercise status (the number of step, exercise time, or the like).

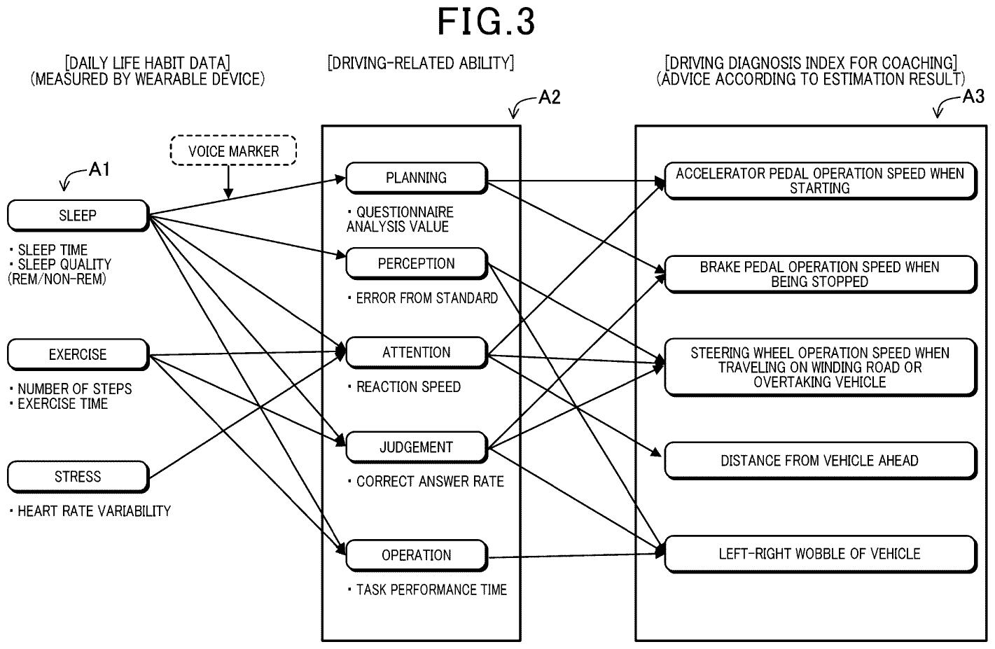

The health level recognition portion 12 recognizes a pre-driving health level of the driver D, based on pre-driving biometric information, which is biometric information recognized by the biometric information recognition portion 11 before the driver D starts driving the vehicle 100. As shown in FIG. 3, the health level recognition portion 12 acquires, as factors that affect the driving-related abilities (planning, perception, attention, judgement, and operation) shown in A2, daily life habit data (sleep, exercise, or stress) of the driver D shown in Al from the biometric information of the driver D, and recognizes the health level of the driver D based on the daily life habit data. In addition, as the daily life habit data, voice data of the driver D acquired by a dialogue application to be executed by the wearable device 160 or the communication terminal 1 may be used as a voice marker to recognize the health level of the driver D from results of voice analysis.

Regarding the driving-related abilities of A2, for example, the planning is evaluated by questionnaire analysis values, the perception is evaluated by an error from a standard, the attention is evaluated by a reaction speed, the judgement is evaluated by a correct answer rate, and the operation is evaluated by a task performance time. Regarding the daily life habit data of A1, the sloop includes a sleep time and a sleep quality (REM sleep, non-REM sleep), the exercise includes the number of steps and an exercise time, and the stress includes heart rate variability. The evaluation of the driving-related abilities is used as a driving diagnosis index for coaching to be notified to the driver D, as shown in A3.

The health level recognition portion 12 recognizes that the health level of the driver D is declining, for example, in the following cases.

Pre-driving state 1: A case of being estimated from the heart rate variability of the driver D that the stress of the driver is increasing.

Pre-driving state 2: A case where the sleep time of the driver D is shorter than the former average.

Pre-driving state 3: A case where the exercise time of the driver D is shorter than the former average.

The driving behavior recognition portion 13 acquires, through communication with the vehicle 100, measurement information Dri regarding the driving behavior of the driver D measured in the vehicle 100 and thus recognizes the driving behavior of the driver D. The measurement information Dri involves an operating speed of the accelerator pedal 141 detected by an accelerator pedal sensor, an operating speed of the brake pedal 142 detected by a brake pedal sensor, an operating speed of the steering wheel detected by a steering sensor, and left-right wobbles of the vehicle 100 detected by a yaw sensor. The driving behavior recognition portion 13 recognizes the driving behavior such as sudden acceleration, sudden deceleration, or abrupt steering, based on the measurement information Dri.

The predicted risk score calculation portion 14 calculates a predicted risk score for the movement of the vehicle 100 driven by the driver D, based on the pre-driving health level of the driver D recognized by the health level recognition portion 12, the heart rate of the driver D, who is driving, recognized by the biometric information recognition portion 11, and the driving behavior of the driver D recognized by the driving behavior recognition portion 13. The process to be executed by the predicted risk score calculation portion 14 will be described below.

The moving body position recognition portion 15 recognizes the position (current position) of the vehicle 100 based on a position detection signal of the GNSS sensor 33. The moving environment recognition portion 16 recognizes a future traveling environment (moving environment) of the vehicle 100 based on the position of the vehicle 100 recognized by the moving body position recognition portion 15 and traffic information Trd (including map information, congestion information, weather information, or the like) transmitted from the traffic information server 210. The traffic information may be acquired through communication with the navigation device 121 provided in the vehicle 100. When the predicted risk score calculated by the predicted risk score calculation portion 14 is equal to or greater than a predetermined value, the moving route guidance portion 17 executes route guidance that notifies the driver of a moving route that provides guidance for a travel route where a driving load is reduced.

### 3. Calculation Process of Predicted Risk Score

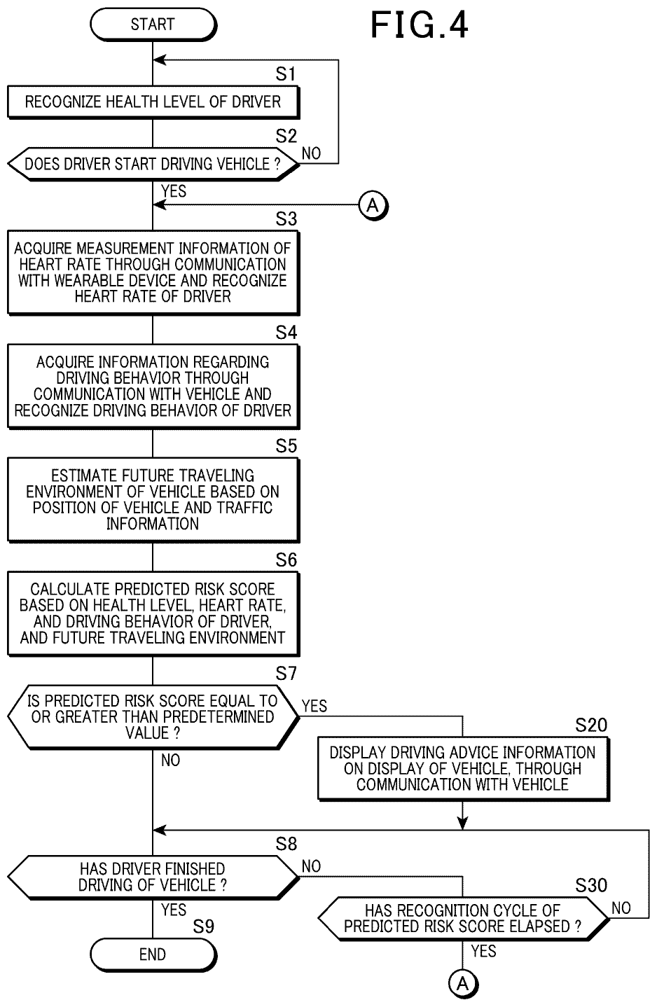

A procedure of a calculation process of a predicted risk score to be executed by the driver monitoring device 2 will be described with reference to flowcharts shown in FIGS. 4.

In steps S1 and S2 in FIG. 4, the health level recognition portion 12 repeatedly executes a process of recognizing a health level of the driver D before the driver D starts driving the vehicle 100 in step S1 until it is recognized in step S2 that the driver D starts driving the vehicle 100, based on the biometric information recognized by the biometric information recognition portion 11 before the driver D starts driving the vehicle 100 as described above. The health level recognition portion 12 recognizes, through the communication with the vehicle 100, that the driver D starts driving the vehicle 100.

In subsequent step S3, the biometric information recognition portion 11 acquires measurement information Bid regarding the heart rate of the driver D through communication with the wearable device 160, and thus recognizes the heart rate of the driver D. In subsequent step S4, the driving behavior recognition portion 13 acquires measurement information Dri regarding the driving behavior through communication with the vehicle 100, and thus recognizes the driving behavior of the driver D.

In subsequent step S5, the moving environment recognition portion 16 recognizes a future traveling environment of the vehicle 100 based on the position of the vehicle 100 recognized by the moving body position recognition portion 15 and traffic information Mad acquired from traffic information server 210. In subsequent step S5, the predicted risk score calculation portion 14 calculates a predicted risk score based on the pre-driving health level, heart rate, and driving behavior of the driver D and the future traveling environment of the vehicle 100.

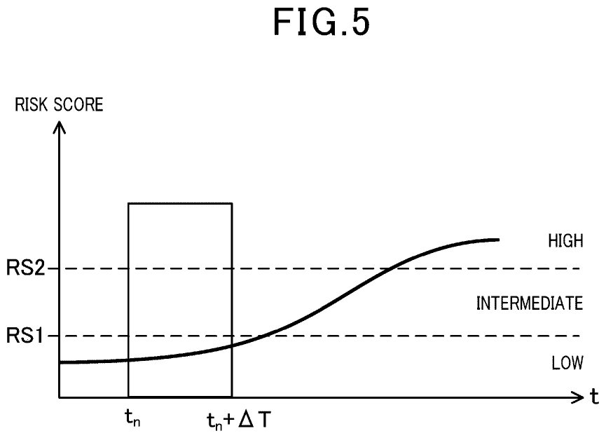

Here, FIG. 5 shows an example of a transition of a risk score by setting a vertical axis to a risk score indicating the degree of risk when the vehicle 100 moves by driving of the driver D and a horizontal axis to a time t. Here, tₙ indicates a time point of calculation of a risk score. The risk score is calculated based on the driving behavior and the heart rate of the driver D, and the fluctuation in the heart rate is an index of the stress of the driver D, but the time constant of the change in the amount of hormones (such as cortisol) secreted in the body of the driver D to cope with stress is low (the secretion amount does not change immediately).

Therefore, the predicted risk score calculation portion 14 calculates, from Formula (1) below, a predicted risk score which is a predicted value of the risk score at tₙ+ΔT, which is a time after ΔT (for example, 15 minutes) from tₙ, assuming that the level of driving behavior indicates a reference value of the risk score.

$$Pd ( {t}_{n} + Δ T ) = α \cdot Da ( {t}_{n} ) \times β \cdot Hb ( {t}_{n} ) + HI \qquad ( 1 )$$

Here, Pd (tₙ+ΔT) represents a predicted risk score after ΔT from tₙ, Da (tₙ) represents a level of driving behavior at tₙ, α and β represent adjustment coefficients, Hb (tₙ) represents a heart rate at tₙ, and HI represents an adjustment value according to a health level of the driver D before driving.

Furthermore, the predicted risk score calculation portion 14 makes compensation such that the higher a driving load dependent on the traveling environment, the higher the predicted risk score becomes, according to the traveling environment of the vehicle 100 at time point tₙ +αT of the vehicle 100 recognized by the moving environment recognition portion 16. The driving load becomes higher when there is heavy traffic on the road, when the road is narrower, and when the weather is bad (rain, strong winds, etc.), for example.

Here, the predicted risk score may be calculated by the predicted risk score calculation portion 14 by applying various statistical models, which are commonly used. For example, the predicted risk score may be calculated by applying statistical processing using a multiple logistic model or the like.

In subsequent step S7, the predicted risk score calculation portion 14 determines whether the predicted risk score is equal to or greater than a predetermined value. When the predicted risk score calculation portion 14 determines that the predicted risk score is equal to or greater than a predetermined value, the process proceeds to step S20, and when the predicted risk score is determined to be less than the predetermined value, the process proceeds to step S8. In step S20, the predicted risk score calculation portion 14 communicates with the vehicle 100 to output driving advice to the driver D according to the predicted risk score by displaying the driving advice on the display 34 or by voice from the speaker 35, and the process proceeds to step S7.

The driving advice includes a route guidance that guides a travel route, which reduces the driving load, from the moving route guidance portion 17. The route guidance is performed by a route display on the display 34 and the output of a guidance voice from the speaker 35.

In step S8, the predicted risk score calculation portion 14 determines, through communication with the vehicle 100, whether the driver D has finished driving the vehicle 100. Then, when the predicted risk score calculation portion 14 determines that the driver D has finished driving the vehicle 100, the process proceeds to step S9, and when the driver D is determined to not have finished driving the vehicle 100, the process proceeds to step S30. In step S30, when the predicted risk score calculation portion 14 determines that the recognition cycle of the predicted risk score has elapsed, the process proceeds to step S3 and the process is executed again from step S3, and when the recognition cycle of the predicted risk score has not elapsed, the process proceeds to step S8.

### 4. Another Embodiment

In the above-described embodiment, the biometric information recognition portion 11 recognizes the heart rate as the biometric information of the driver D. In another embodiment, the biometric information recognition portion 11 may recognize, as the biometric information of the driver D, the blood pressure, the body temperature, or the like of the driver D detected by a vital sensor provided in the wearable device 160. In addition, the biometric information recognition portion 11 may acquire detection information regarding the heart rate, the blood pressure, the body temperature, or the like detected by a vital sensor provided in the communication terminal 1 or the vehicle 100, to recognize the biometric information of the driver D.

In the above-described embodiment, the health level recognition portion 12 is provided, and the predicted risk score calculation portion 14 calculates the predicted risk score based on the pre-driving health level of the driver D recognized by the health level recognition portion 12. In another embodiment, the health level recognition portion 12 may not be provided.

In the above-described embodiment, the moving body position recognition portion 15 and the moving environment recognition portion 16 are provided, and the predicted risk score calculation portion 14 compensates the predicted risk score based on the driving load due to the future traveling environment of the vehicle 100. In another embodiment, the moving body position recognition portion 15 and the moving environment recognition portion 16 may not be provided, and the predicted risk score may not be compensated based on the driving load due to the future traveling environment of the vehicle 100.

In the above-described embodiment, the moving route guidance portion 17 is provided to guide the driver D to the travel route that reduces the driving load on the driver D when the predicted risk score equal to or greater than a predetermined value is calculated, but the moving route guidance portion 17 may not be provided, whereby the above guidance may not be performed.

In the above-described embodiment, the driver monitoring device 2 is configured as a function of the communication terminal 1. In another embodiment, the driver monitoring device of the present disclosure may be configured as a function of an in-vehicle device such as ECU 110 provided in the vehicle 100, or as a dedicated in-vehicle device. In this case, the in-vehicle device communicates with the wearable device 160 worn by the driver D to receive biometric information measurement information Bid and to recognize biometric information.

The driver monitoring device of the present disclosure may be configured as a function of a server such as the vehicle management server 211. In this case, the server receives biometric information measurement information Bid and recognizes biometric information through communication with the wearable device 160 worn by the driver D (direct communication or communication via the communication terminal 1 or the vehicle 100).

FIG. 2 is a schematic diagram illustrating the configuration of the driver monitoring device 2 by dividing the configuration according to main process contents to facilitate understanding of the present invention, and the configuration of the driver monitoring device 2 may be divided according to other categories. The process of each of the components may be executed by one hardware unit, or may be executed by a plurality of hardware units. Further, the process of each of the components illustrated in FIG. 4 may be executed by one program, or may be executed by a plurality of programs.

### 5. Configuration Supported by Embodiment

The above-described embodiment is a specific example of the following configuration.

(Configuration 1) A driver monitoring device including: a biometric information recognition portion that recognizes biometric information of a driver of a moving body; a driving behavior recognition portion that recognizes driving behavior of the driver, who is driving the moving body; and a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.

According to the driver monitoring device of Configuration 1, it is possible to calculate an index that predicts future changes in the risk of the driving situation with which the driver is confronted.

(Configuration 2) In the driver monitoring device according to Configuration 1, the driver monitoring device further includes a health level recognition portion that recognizes a health level of the driver, based on pre-driving biometric information, which is the biometric information of the driver, recognized by the biometric information recognition portion before the driver starts driving the moving body, and the predicted risk score calculation portion calculates the predicted risk score, based on the driving behavior of the driver recognized by the driving behavior recognition portion at the predetermined time point, the on-driving biometric information recognized by the biometric information recognition portion at the predetermined time point, and the health level of the driver recognized by the health level recognition portion.

According to the driver monitoring device of Configuration 2, it is possible to improve the accuracy of the predicted risk score by reflecting the pre-driving health level of the driver.

(Configuration 3) In the driver monitoring device according to Configuration 1 or 2, the biometric information recognition portion recognizes, as the biometric information of the driver, a heart rate of the driver.

According to the driver monitoring device of Configuration 3, it is possible to calculate the predicted risk score based on the heart rate of the driver which varies depending on the stress level of the driver.

(Configuration 4) In the driver monitoring device according to any one of Configurations 1 to 3, the driver monitoring device further includes: a moving body position recognition portion that recognizes a position of the moving body; and a moving environment recognition portion that recognizes a moving environment of the moving body after the predetermined time point, based on the position of the moving body at the predetermined time point recognized by the moving body position recognition portion, and the predicted risk score calculation portion compensates the predicted risk score based on the moving environment estimated by a moving environment estimation portion.

According to the driver monitoring device of Configuration 4, it is possible to compensate the predicted risk score in consideration of the moving environment (the degree of congestion on the road, narrowness of the road, and weather when the moving body is a vehicle) that affects the degree of risk when the driver drives the moving body.

(Configuration 5) In the driver monitoring device according to Configuration 4, the driver monitoring device further includes a moving route guidance portion that guides a moving route that is a moving environment recognized by the moving environment recognition portion to reduce a driving load of the driver when the predicted risk score calculation portion calculates the predicted risk score that is equal to or greater than a predetermined value.

According to the driver monitoring device of Configuration 5, when the predicted risk score is equal to or greater than a predetermined value, the risk level of moving by the moving body can be reduced by guiding the moving route that reduces the driving load and prompting the driver to change the moving route.

(Configuration 6) A driver monitoring method to be executed by a computer, the driver monitoring method including: a biometric information recognition step of recognizing biometric information of a driver of a moving body; a driving behavior recognition step of recognizing driving behavior of the driver, who is driving the moving body; and a predicted risk score calculation step of calculating, based on driving behavior of the driver recognized in the driving behavior recognition step at a predetermined time point while the driver is driving the moving body and on-driving biometric information, which is biometric information of the driver, recognized in the biometric information recognition step at the predetermined time point, a predicted risk score indicating a risk level regarding movement of the moving body due to driving of the driver after the predetermined time point.

The computer executes the driver monitoring method of Configuration 6, whereby it is possible to obtain the same operational effect as the driver monitoring device of Configuration 1.

(Configuration 7) A non-transitory computer-readable storage medium storing a program causing a computer to function as: a biometric information recognition portion that recognizes biometric information of a driver of a moving body; a driving behavior recognition portion that recognizes driving behavior of the driver, who is driving the moving body; and a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.

The computer executes the program of Configuration 7, whereby it is possible to implement the configuration of the driver monitoring device of Configuration 1.

### REFERENCE SIGNS LIST

1 communication terminal
2 driver monitoring device
10 processor
11 biometric information recognition portion
12 health level recognition portion
13 driving behavior recognition portion
14 predicted risk score calculation portion
15 moving body position recognition portion
16 moving environment recognition portion
17 moving route guidance portion
21 program
30 communication unit
31 camera
32 acceleration sensor
33 GNSS sensor
34 display
35 speaker
36 microphone
100 vehicle
110 ECU
120 communication unit
121 navigation device
130 front camera
131 driver monitor camera
132 display
133a, 133b speaker
150 terminal holder
160 wearable device
200 communication network
210 traffic information server
211 vehicle management server
D driver

1. A driver monitoring device comprising:
a biometric information recognition portion that recognizes biometric information of a driver of a moving body;
a driving behavior recognition portion that recognizes driving behavior of the driver, who is driving the moving body; and
a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.

2. The driver monitoring device according to claim 1, further comprising
a health level recognition portion that recognizes a health level of the driver, based on pre-driving biometric information, which is the biometric information of the driver, recognized by the biometric information recognition portion before the driver starts driving the moving body, wherein
the predicted risk score calculation portion calculates the predicted risk score, based on the driving behavior of the driver recognized by the driving behavior recognition portion at the predetermined time point, the on-driving biometric information recognized by the biometric information recognition portion at the predetermined time point, and the health level of the driver recognized by the health level recognition portion.

3. The driver monitoring device according to claim 1, wherein
the biometric information recognition portion recognizes, as the biometric information of the driver, a heart rate of the driver.

4. The driver monitoring device according to claim 1, further comprising:
a moving body position recognition portion that recognizes a position of the moving body; and
a moving environment recognition portion that recognizes a moving environment of the moving body after the predetermined time point, based on the position of the moving body at the predetermined time point recognized by the moving body position recognition portion, wherein
the predicted risk score calculation portion compensates the predicted risk score based on the moving environment estimated by a moving environment estimation portion.

5. The driver monitoring device according to claim 4, further comprising
a moving route guidance portion that guides a moving route that is a moving environment recognized by the moving environment recognition portion to reduce a driving load of the driver when the predicted risk score calculation portion calculates the predicted risk score that is equal to or greater than a predetermined value.

6. A driver monitoring method to be executed by a computer, the driver monitoring method comprising:
a biometric information recognition step of recognizing biometric information of a driver of a moving body;
a driving behavior recognition step of recognizing driving behavior of the driver, who is driving the moving body; and
a predicted risk score calculation step of calculating, based on driving behavior of the driver recognized in the driving behavior recognition step at a predetermined time point while the driver is driving the moving body and on-driving biometric information, which is biometric information of the driver, recognized in the biometric information recognition step at the predetermined time point, a predicted risk score indicating a risk level regarding movement of the moving body due to driving of the driver after the predetermined time point.

7. A non-transitory computer-readable storage medium storing a program causing a computer to function as:
a biometric information recognition portion that recognizes biometric information of a driver of a moving body;
a driving behavior recognition portion that recognizes driving behavior of the driver, who is driving the moving body; and
a predicted risk score calculation portion that calculates, based on driving behavior of the driver recognized by the driving behavior recognition portion at a predetermined time point while the driver is driving the moving body and on-driving biometric information, which is biometric information of the driver, recognized by the biometric information recognition portion at the predetermined time point, a predicted risk score indicating a risk level regarding movement of the moving body due to driving of the driver after the predetermined time point.
